(19) United States
(12) Patent Application Publication
(10) Pub. No.: US 2026/0098068 A1
(43) Pub. Date: Apr. 9, 2026

(54) GC-GLOBULIN FOR USE IN TREATING DIABETES

(71) Applicant: The University of Birmingham, Edgbaston, Birmingham (GB)

(72) Inventors: DAVID HODSON, Headington (GB); Katrina VILORIA, Headington (GB); Martin HEWISON, Edgbaston, Birmingham (GB)

(21) Appl. No.: 19/114,441

(22) PCT Filed: Sep. 22, 2023
(86) PCT No.: PCT/GB2023/052453

§ 371 Date: Mar. 22, 2025

(30) Foreign Application Priority Data
Sep. 22, 2022 (GB) 2213863.0

(51) Int. Cl. C07K 14/47; A61K 38/00; A61P 3/10; A61P 3/14

(52) U.S. Cl. CPC C07K 14/4717; A61P 3/10; A61P 3/14; A61K 38/00

(57) ABSTRACT

The invention relates to GC-globulin, or a variant thereof, for use in treating diabetes.

The present invention relates to a novel agent for treating diabetes.

To ensure normal body function, blood glucose levels must be tightly controlled. This glucose homeostasis is achieved predominantly by the blood sugar-lowering hormone insulin and the blood sugar-raising hormone glucagon, both of which are secreted by cells in the pancreas. Disturbances in the interplay of these hormone can lead to disorders such as diabetes, whose prevalence and co-morbidities place significant strain on healthcare services.

Diabetes is a serious condition that occurs because blood glucose levels are too high. There are two main types of diabetes: type 1 and type 2. In type 1 diabetes, the immune system destroys insulin-secreting beta cells in the pancreas, meaning that the body cannot produce any insulin. In type 2 diabetes, the body produces insulin, but it does not make enough or does not use it well. In both type 1 and type 2 diabetes, too much glucose stays in the blood, and not enough reaches the cells.

Treatment for type 1 diabetes involves taking insulin or receiving an islet transplant to restore endogenous insulin production. For type 2 diabetes, a number of treatments exist, the most recent being the glucagon-like peptide-1 receptor agonist (GLP1RA) class. GLP1RA stimulates insulin secretion in a glucose-dependant manner, i.e. only when blood sugar levels are high. Whilst this treatment works well, there is still a need to develop drugs which lead to further improvements in insulin secretion and pancreatic beta cell mass.

In addition to absent or reduced insulin secretion, patients with type 1 and type 2 diabetes often present with episodes of too low blood glucose (hypoglycemia). Hypoglycemia occurs because counter-regulation responses are absent when endogenous insulin is not secreted in sufficient quantities (or at all). Hypoglycemia is dangerous and is the major cause of death in type 1 diabetic patients. It can also cause fainting and permanent changes to the brain. Despite this, no drug therapy can yet tackle hypoglycemia, although treatments (e.g. somatostatin antagonists) are in clinical trials.

Most patients treated with GLP1RA will end up requiring treatment escalation and there is therefore an appetite to develop new drugs with better efficacy at preserving insulin-secreting beta cells. Moreover, hypoglycemia remains a major issue for type 1 and type 2 diabetic patients.

Accordingly, in one aspect of the present invention, there is provided GC-globulin, or a variant thereof, for use in treating diabetes in a subject. The variants may include C-terminal modifications of GC-globulin, N-terminal modifications, PEGylation, fatty acid side chain additions or alkyl side chain additions.

Group-specific component of serum (GC-globulin or GC), also known as vitamin D-binding protein, is an ˜58kDa protein that transports vitamin D metabolites and fatty acids in the circulation¹⁻². GC-globulin is also amongst the most potent actin scavengers in the body and acts in concert with gelsolin to sequester actin filaments released from lysed cells³⁻⁴. GC/Gc, the gene encoding GC, was thought to be almost exclusively expressed in the liver, where sterol derivatives such as cholecalciferol are converted into pre-hormone 25-OH vitamin D (25(OH)D)⁵. However, recent studies have shown that human and mouse alpha cells express equally high levels of GC/Gc⁶⁻⁷, whereas the gene is absent from beta cells under normal conditions⁷⁻⁸.

Previous studies have shown that the gene encoding GC-globulin has open chromatin regions pointing to its role as an alpha cell signature gene (i.e. it is highly alpha cell specific in the pancreatic islet and can be used to identify alpha cells). Alpha cells lacking GC-globulin show a number of derangements including: smaller size, impaired Ca²⁺ and Na⁺ fluxes in response to low glucose, as well as dysregulated glucagon secretion. Moreover, GC-globulin levels have been shown to be reduced in alpha cells of pancreas samples from donors with type 1 diabetes.

In the present application, GC-globulin was studied in the context of metabolic stress resembling pre-diabetes and type 2 diabetes. In a preclinical mouse model of type 2 diabetes, deletion of GC-globulin led to impairments in alpha cell function, including elevated basal glucagon release and impaired low glucose-stimulated glucagon secretion. Unexpectedly, since a specific uptake transporter is required, GC-globulin levels could be restored in alpha cells by applying exogenous GC-globulin. This was found to be due to expression of megalin/cubulin in the pancreatic islet, which are known GC-globulin transporters. Restoring GC-globulin levels led to restoration of alpha cell functional defects including glucagon secretion. Unexpectedly it was found that GC-globulin supplementation led to a large increase in glucose-stimulated and GLP1RA-stimulated insulin secretion, possibly via direct or indirect effects. Lastly, samples from donors with type 2 diabetes showed that the normal positive relationship between GC-globulin and glucagon expression was lost.

Together, these findings show that: 1) GC-globulin is lacking during type 1 and type 2 diabetes; 2) GC-globulin is required for normal, regulated glucagon and insulin secretion; 3) administration of exogenous GC-globulin restores cellular levels of the protein; and 4) treatment with GC-globulin rescues disturbances in glucagon and insulin secretion, restoring normal glucose levels.

In another aspect of the present invention, there is provided a pharmaceutical composition for use in treating diabetes in a subject, comprising GC-globulin or a variant thereof and a pharmaceutically active carrier.

In another aspect of the present invention, there is provided a method of treating diabetes in a subject in need thereof, comprising administering GC-globulin or a variant thereof to the subject.

In another aspect of the present invention, there is provided a use of GC-globulin or a variant thereof for the manufacture of a medicament for the treatment of diabetes.

In one embodiment of any one of the above aspects, the diabetes is type 1 diabetes and/or type 2 diabetes.

In one embodiment of any one of the above aspects, GC-globulin is administered orally, subcutaneously or intramuscularly. The GC-globulin may be formulated for oral, subcutaneous or intramuscular administration.

In one embodiment of any one of the above aspects, GC-globulin is present in an amount of about 1 mg/kg to about 1000 mg/kg, or about 5 mg/kg to about 500 mg/kg, or about 25 mg/kg to about 250 mg/kg, or about 50 mg/kg to about 375 mg/kg, or about 75 mg/kg to about 250 mg/kg, or about 100 mg/kg to about 200 mg/kg. one embodiment of any of the above aspects, GC-globulin may be administered once or twice daily. one embodiment of any one of the above aspects, the subject is human.

The skilled person will appreciate that preferred features of any one embodiment and/or aspect of the invention may be applied to other embodiments and/or aspects of the invention,

There now follows by way of example only a detailed description of the present invention with reference to the accompanying drawings, in which:

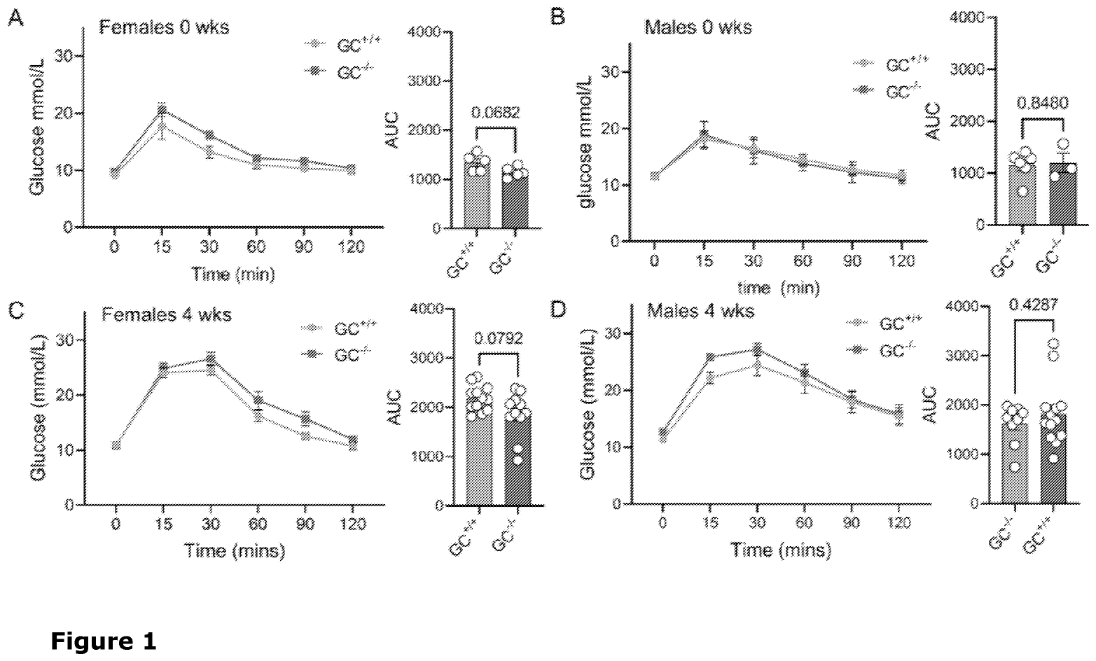

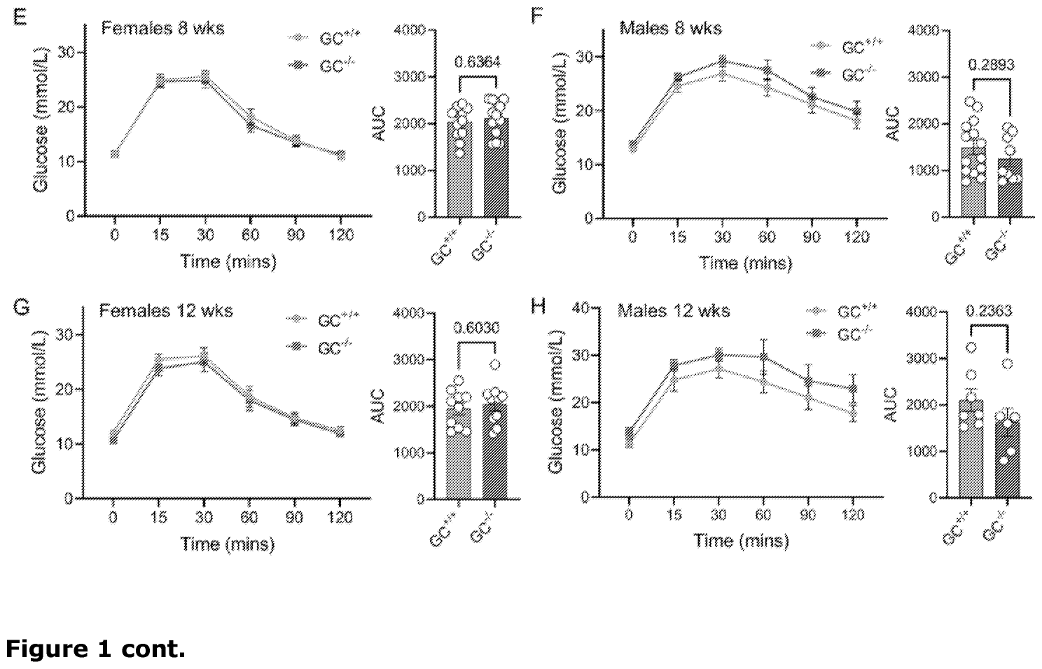

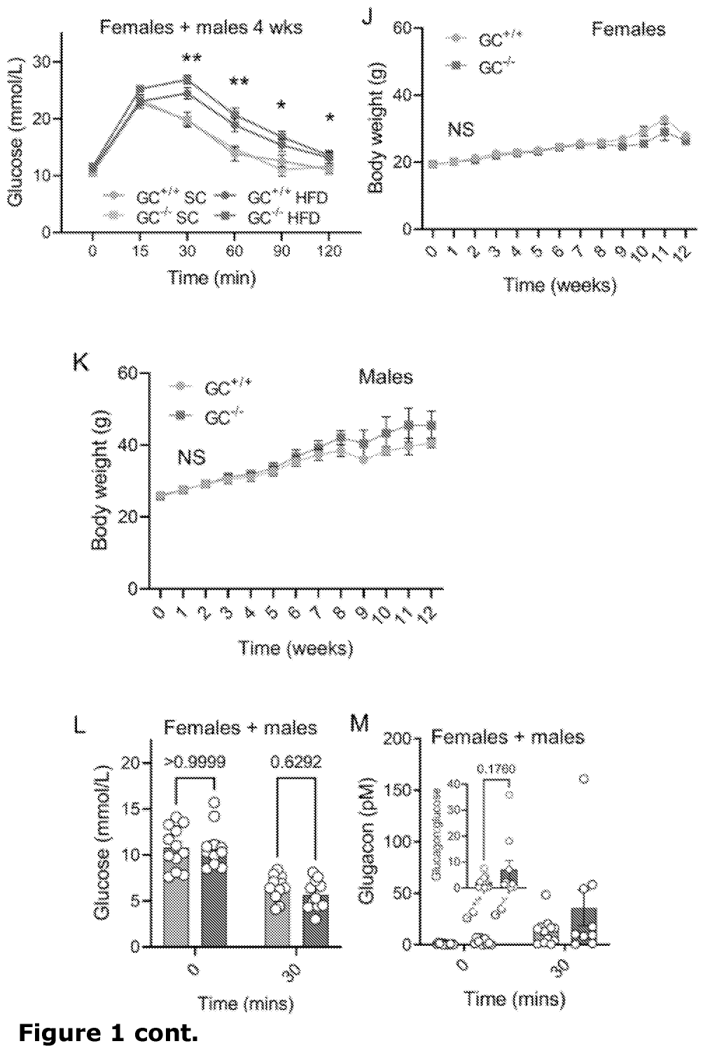

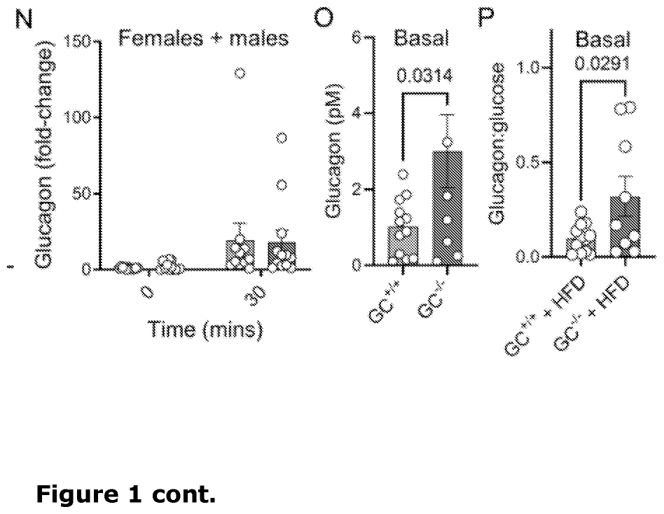

FIG. 1 illustrates the glucose tolerance, body weight and glucagon secretion in GC^{+/+} and GC^{-/-} mice during high fat diet. FIGS. 1A-H) Intraperitoneal glucose tolerance is similar in female and male GC^{+/+} and GC^{-/-} mice during 0 weeks (A, B), 4 weeks (C, D), 8 weeks (E, F) and 12 weeks (G, H) HFD, as shown by bar graphs and AUC (0 weeks, n=3-6 animals; 4 weeks, n=9-13 animals; 8 weeks, n=9-14 animals; 12 weeks, n=6-10 animals) (two-way RM ANOVA with Sidak post-hoc test for line graphs; Mann-Whitney test or unpaired t-test for AUC). FIG. 1I) Pooled data from age-matched male and female mice fed SC or HFD for 4 weeks (n=5-25 mice) (two-way RM ANOVA with Tukey's post-hoc test). FIGS. 1J and 1K) Body weight gain is similar in female (J) and male (K) GC^{+/+} and GC^{-/-} mice during 0-12 weeks HFD (two-way ANOVA with Sidak post-hoc test). FIG. 1L) Glucose responses to intraperitoneal injection of insulin, used to stimulate glucagon release, are not significantly different in GC^{+/+} and GC^{-/-} mice at 0 mins and 30 mins (n=11-12 animals) (two-way RM ANOVA with Sidak post-hoc test). FIGS. 1M and 1N) Basal but not insulin-stimulated plasma glucagon concentrations are significantly higher in GC^{-/-} versus GC^{+/+} mice, shown by raw values (M) fold-change (N) (n=9-12 animals) (unpaired t test) (inset in M is glucagon: glucose ratio). FIGS. 1O and 1P) Basal glucagon levels from (M) shown in a separate graph for clarity, alongside glucagon: glucose ratio at t=30 mins post insulin injection (P). Glucose and glucagon readings in L-P are from the same mice, albeit with dropout of two samples in which glucagon was undetectable by ELISA. AUC, area-under-the-curve. SC, standard chow; HFD, high-fat diet.

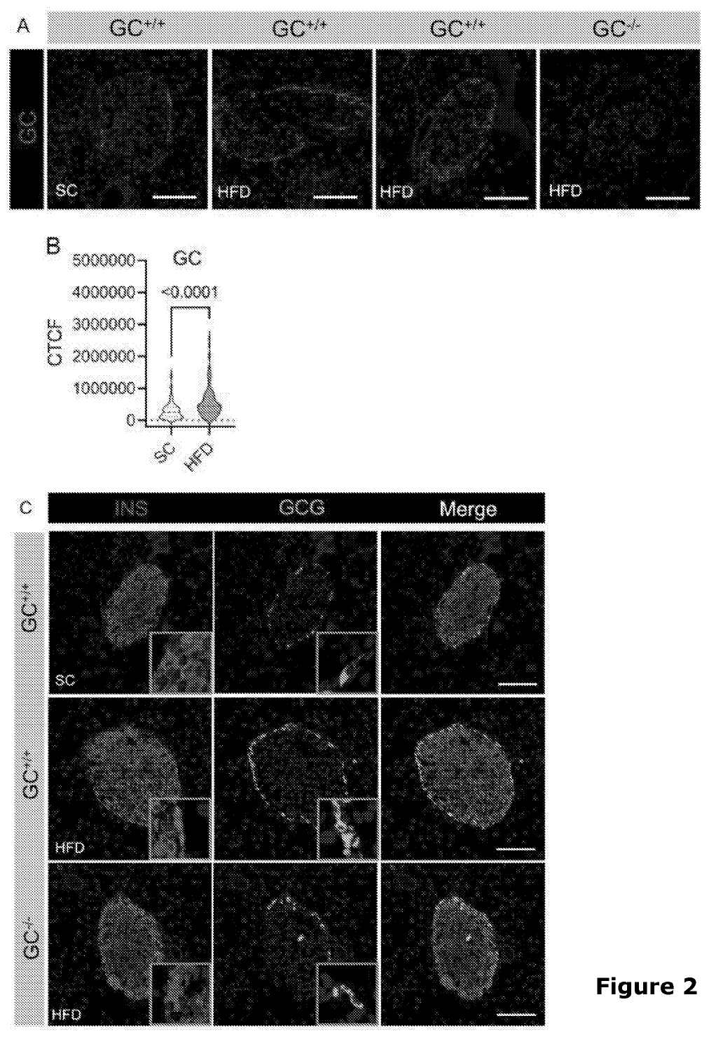

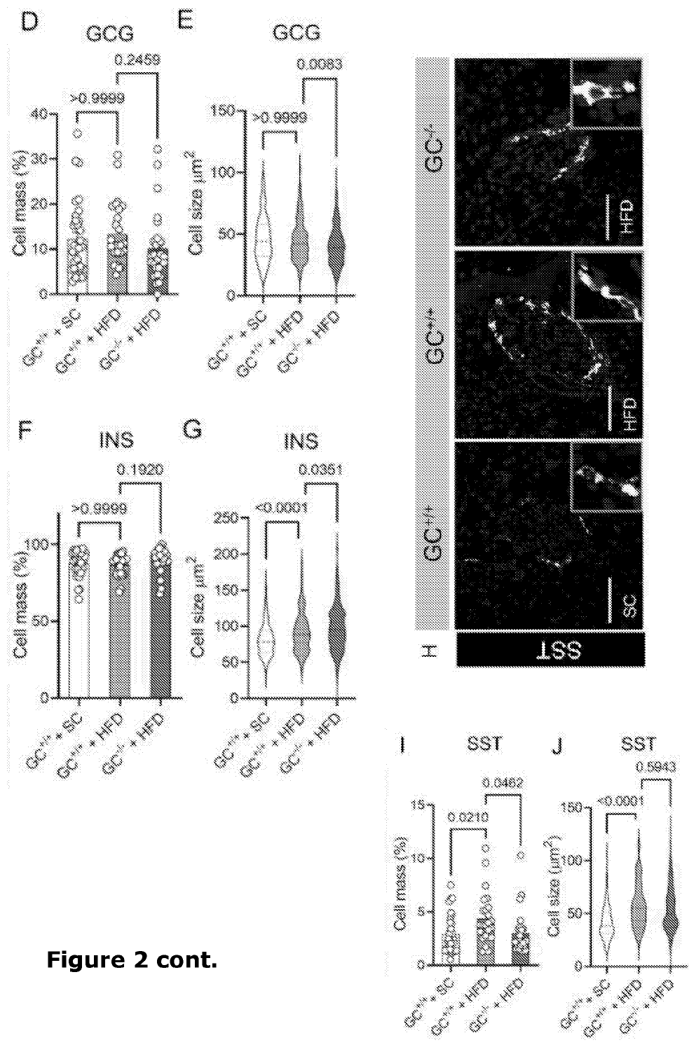

FIG. 2 illustrates α cell, β cell and δ cell morphometry in GC^{+/+} and GC^{-/-} mice during high fat diet. FIGS. 2A, 2B) GC expression levels are increased 2-fold following 8 weeks HFD compared to SC (A; left two image panels), quantified in (B) using corrected total cell fluorescence (CTCF). Note that immunopositivity is detected in HFD-fed GC^{+/+} and not GC^{-/-} mice, thus validating the antibody under the conditions used here (A; right two image panels). FIGS. 2C-G) Proportion α cells per islet (C, D) (n=26-32 islets from 3-4 mice), as well as α cell size (C, E) (n=233-301 cells from 3-4 mice), is not affected by 8 weeks HFD in GC^{+/+} mice. Deletion of GC (GC^{-/-}) leads to fewer and smaller α cells per islet (C-E) (proportion α cell per islet, n=26-32 islets from 4 mice) (α cell size, n=233-301 cells from 3-4 mice). HFD (8 weeks) does not increase proportion β cells per islet (C, F) (n=26-32 islets from 3-4 mice), but increases β cell size, an effect accentuated following deletion of GC (GC^{-/-}) (C, G) (proportion α cells per islet, n=26-32 islets from 3-4 mice) (β cell size, n=240-320 cells from 3-4 mice) (one-way ANOVA with Bonferroni's post-hoc test). FIGS. 2H-J) HFD (8 weeks) increases proportion δ cells per islets, as well as δ cell size in GC^{+/+} but not GC^{-/-} islets (GC^{-/-}) (proportion δ cells per islet, n=24-31 islet from 3-4 mice) (δ cell size, n=117-294 cells from 3-4 mice) (one-way ANOVA with Bonferroni's post-hoc test). Scale bar=85 μm. GC, GC-globulin; GCG, glucagon; INS, insulin; SST, somatostatin; SC, standard chow; HFD, high-fat diet.

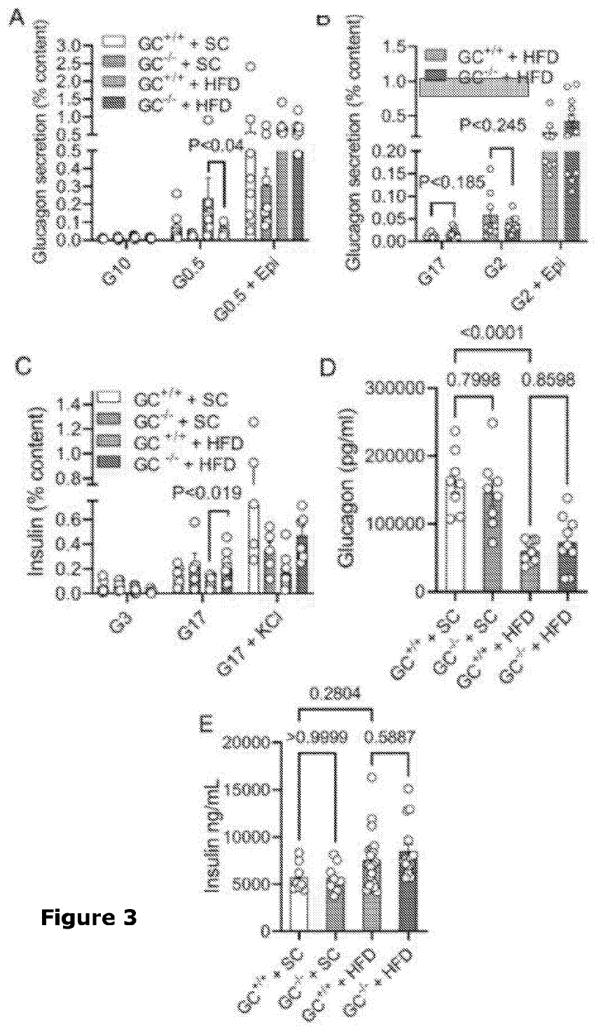

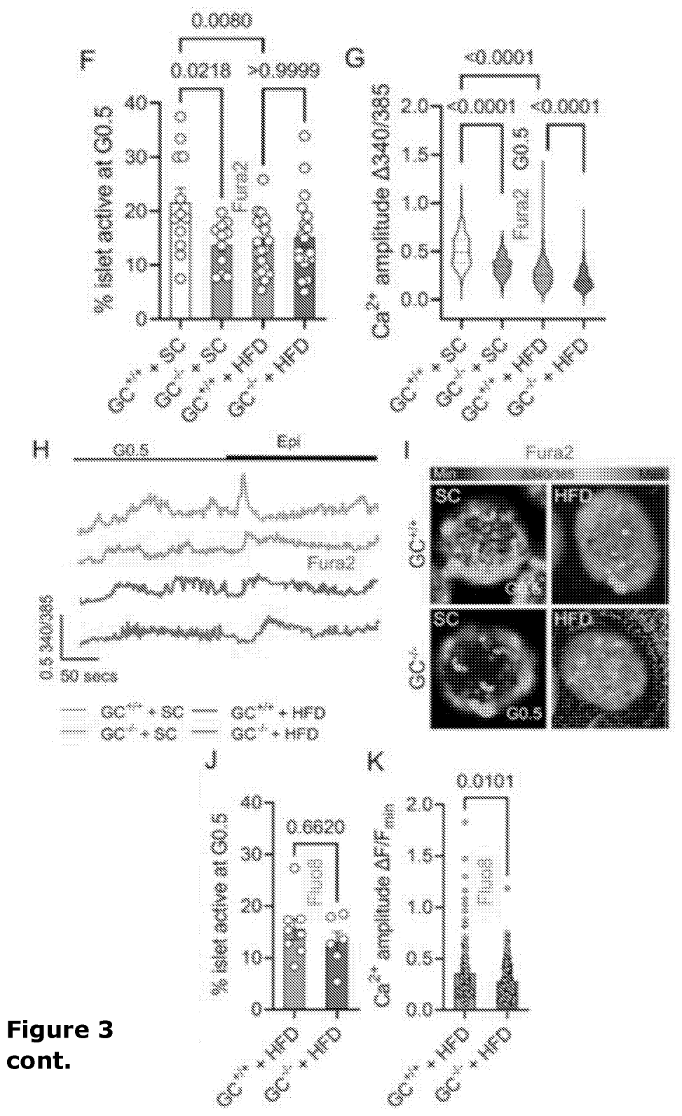

FIG. 3 illustrates hormone secretion and ionic fluxes in islets isolated from high fat diet-fed GC^{+/+} and GC^{-/-} mice. FIG. 3A) Low glucose (0.5 mM)-stimulated glucagon secretion is impaired in SC and HFD islets isolated from GC^{-/-} versus GC^{+/+} mice (SC, n=4-5 animals; HFD, n=7 animals) (Mann-Whitney test). FIG. 3B) As for A) but showing a tendency toward elevated basal glucagon secretion and impaired low glucose-stimulated glucagon secretion in HFD islets from GC^{-/-} versus GC^{+/+} mice (n=3-4 animals) (Student's t-test). FIG. 3C) Glucose-stimulated insulin secretion is significantly increased in GC^{-/-} islets isolated from HFD- but not SC-fed animals (SC, n=7-8 replicates from 3-5 animals; HFD, n=7-8 replicates from 4-5 animals) (Mann-Whitney test). FIGS. 3D and 3E) Total glucagon (D) and insulin (E) concentration is similar in GC^{+/+} and GC^{-/-} islets isolated from SC and HFD-fed mice (n=4-8 animals) (two-way ANOVA with Bonferroni's post-hoc test). FIG. 3F) Proportion of α cells active at low glucose (0.5 mM) was reduced in GC^{-/-} islets from SC-fed but not HFD-fed mice (versus GC^{+/+} littermate controls) (n=11-19 islets from 3-4 animals) (one-way ANOVA with Bonferroni's post-hoc test). FIGS. 3G-I) Amplitude of Ca²⁺ spikes (at 0.5 mM glucose) was reduced in GC^{-/-} versus GC^{+/+} islets from mice on SC. HFD-alone reduced Ca²⁺ spike amplitude and baseline Ca²⁺ concentration, an effect accentuated by deletion of GC (GC^{-/-}). Bar graphs (G) show summary data, traces (H) and images (I) are representative (one-way ANOVA with Bonferroni's post-hoc test) (n=184-339 cells from 3-4 animals). FIGS. 3J and 3K) As for F and G, but using the non-ratiometric Ca²⁺ indicator, Fluo8 (J, n=6-8 islets from 2-3 animals; K, n=50-79 cells from 2-3 animals) (Mann-Whitney test). GC, GC-globulin; G0.5, 0.5 mM glucose; G2, 2 mM glucose; G3, 3 mM glucose, G10, 10 mM glucose; G17, 17 mM glucose. SC, standard chow; HFD, high-fat diet.

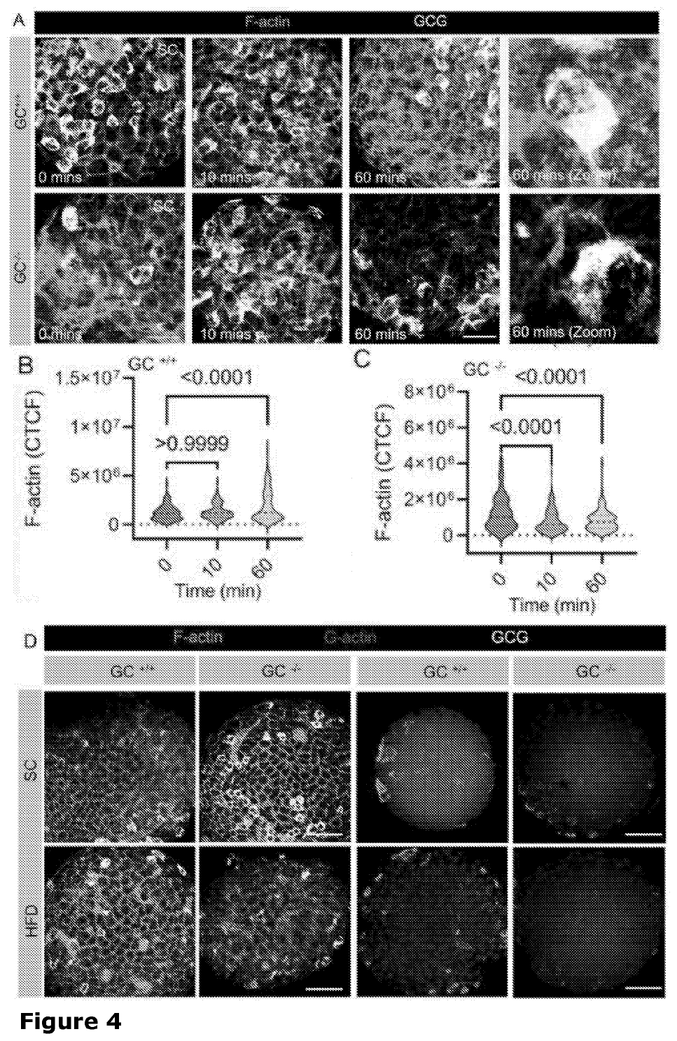

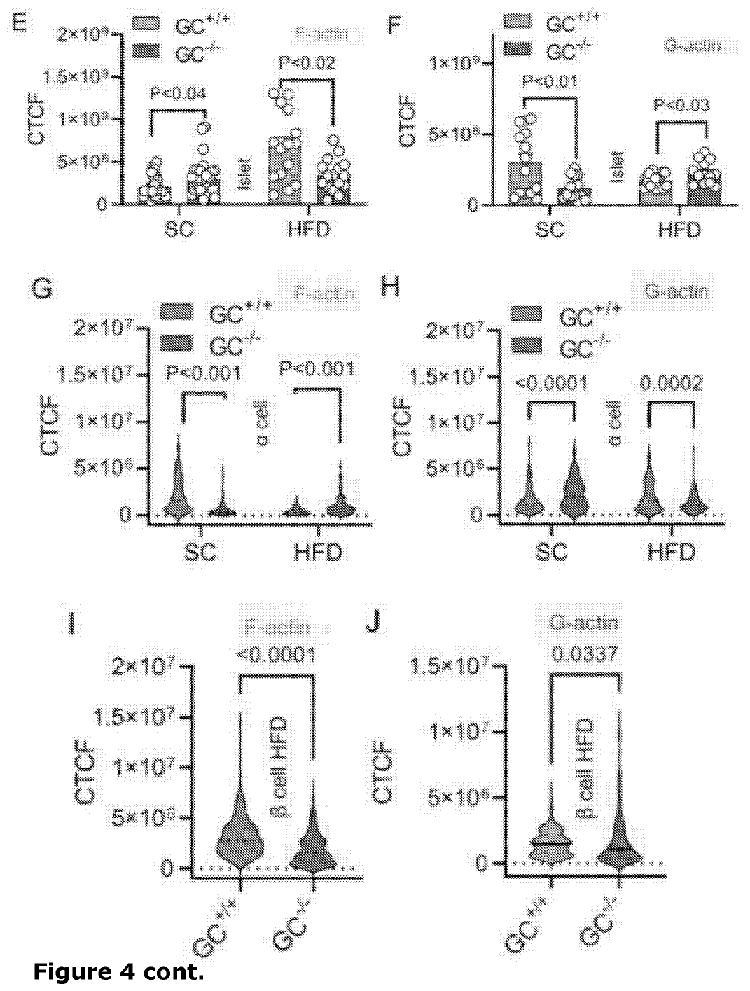

FIG. 4 illustrates F-actin and G-actin levels in islets isolated from high fat diet-fed GC^{+/+} and GC^{-/-} mice. FIGS. 4A-C) Polymeric actin (F-actin) levels increase and decrease in SC GC^{+/+} and GC^{-/-} islets, respectively, following 60 mins stimulation with low (0.5 mM) glucose concentration, as shown by representative images (A) and summary bar graphs (B and C) (n=110-145 α cells from 3 animals) (one-way ANOVA with Bonferroni's post-hoc test) (scale bar=34 μm). FIGS. 4D-F) F-actin density is increased in GC^{-/-} islets from SC-fed mice. HFD induces a large increase in F-actin density in GC^{+/+} islets, which can be partly reversed by deletion of GC (GC^{-/-} ). Monomeric G-actin, which is liberated following F-actin disassembly, shows the opposite trend. Representative images show F-actin and G-actin levels in the islet (D), analysed in (E and F) using corrected total cell fluorescence (CTCF) (Mann-Whitney test or unpaired t test) (n=15-30 islets from 3 animals) (scale bar=53 μm). G-J), As for D-F, but CTCF analysis of F-actin and G-actin in α cells (FIG. 4G and H) and β cells (FIGS. 4I and 4J) (two-way ANOVA with Bonferroni's post-hoc test) (n=159-176 from 3 animals). SC, standard chow; HFD, high-fat diet.

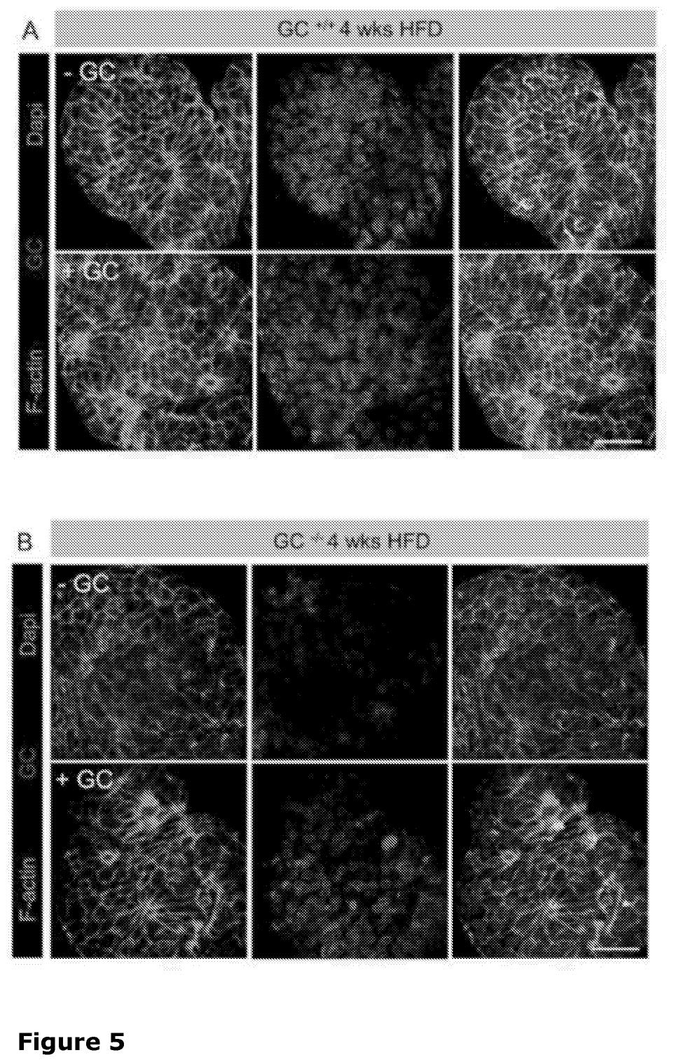

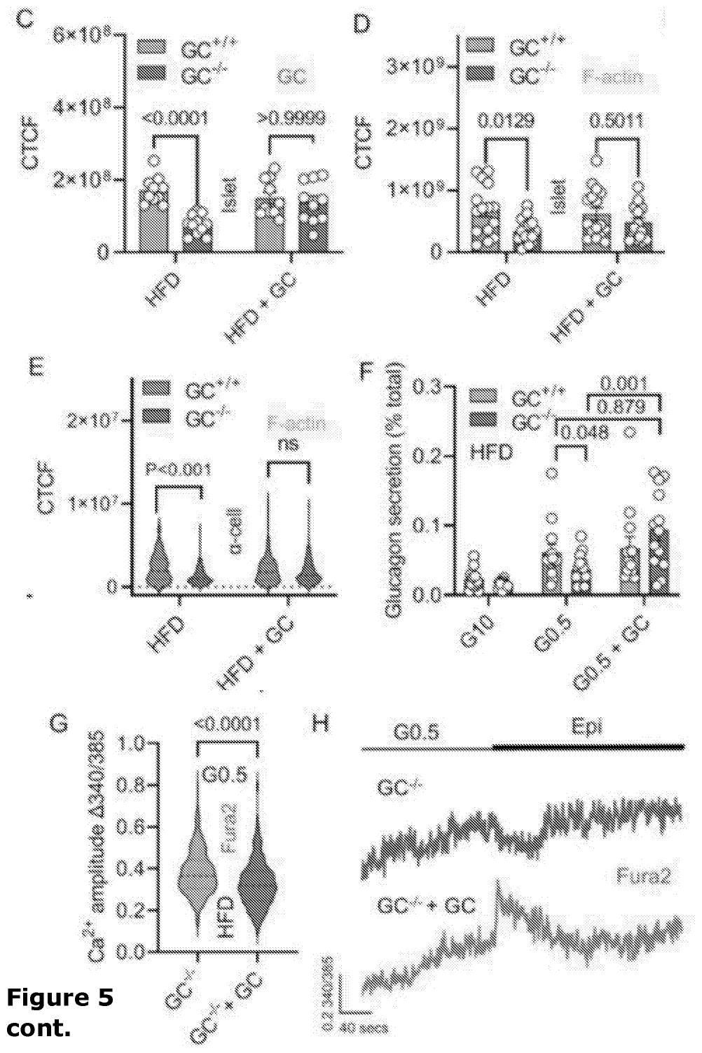

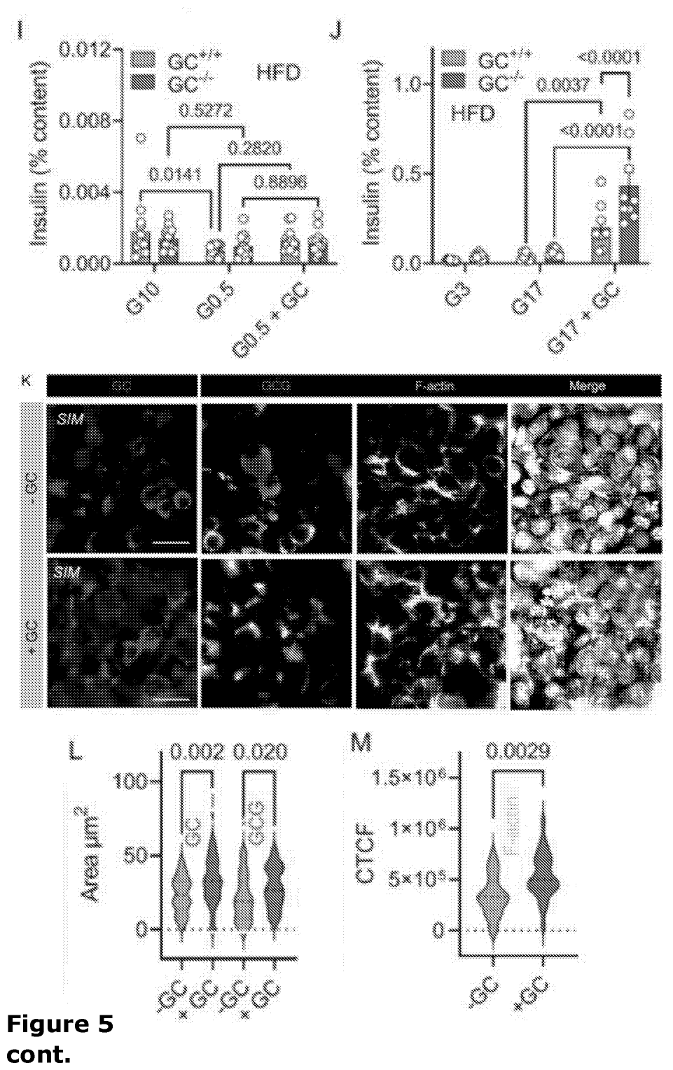

FIG. 5 illustrates the effects of exogenous GC supplementation in islets from high fat diet-fed GC^{+/+} and GC^{-/-} mice. FIGS. 5A and 5B) Representative images showing that GC expression and F-actin levels can only be modified in HFD GC^{-/-} islets following incubation with GC (scale bar=53μm). FIGS. 5C and 5D) Corrected total cell fluorescence (CTCF) analysis showing a significant increase in GC (C) and F-actin (D) levels in GC-treated GC^{-/-}, but not GC^{+/+}, islets (n=10-11 islets from 3 animals) (two-way ANOVA with Bonferroni's post-hoc test). FIG. 5E) As D, but F-actin levels in individual a cells (n=10-11 islets from 3 animals) (two-way ANOVA with Bonferroni's post-hoc test). FIG. 5F) Exogenous GC restores low glucose (G0.5)-stimulated glucagon secretion in GC^{-/-} islets (n=14-16 repeats from 5-7 animals) (Mann-Whitney test). FIGS. 5G and 5H) Exogenous GC does not affect low glucose (G0.5)-stimulated Ca²⁺ rises in GC^{-/-} islets, shown by amplitude (G) and representative traces (H) (n=174-205 cells from 4 animals). FIG. 5I) HFD GC^{-/-} islets fail to shut off insulin secretion at low glucose, although GC treatment itself is unable to influence basal insulin release (n=14 repeats from 4 animals). FIG. 5J) GC treatment amplifies glucose-stimulated insulin secretion, with a larger effect in GC^{-/-} compared to GC^{+/+} islets (n=9 repeats from 2-3 animals) (two-way ANOVA with Bonferroni's post-hoc test). FIGS. 5K-M) GC levels can be supplemented in human islets (K, L), leading to an increase in glucagon granule area (L) and F-actin density (M). Note that the GC images are not from the same islets as those showing GCG and F-actin. Scale bar=15 μm. GC, GC-globulin; G0.5, 0.5 mM glucose; HFD, high-fat diet.

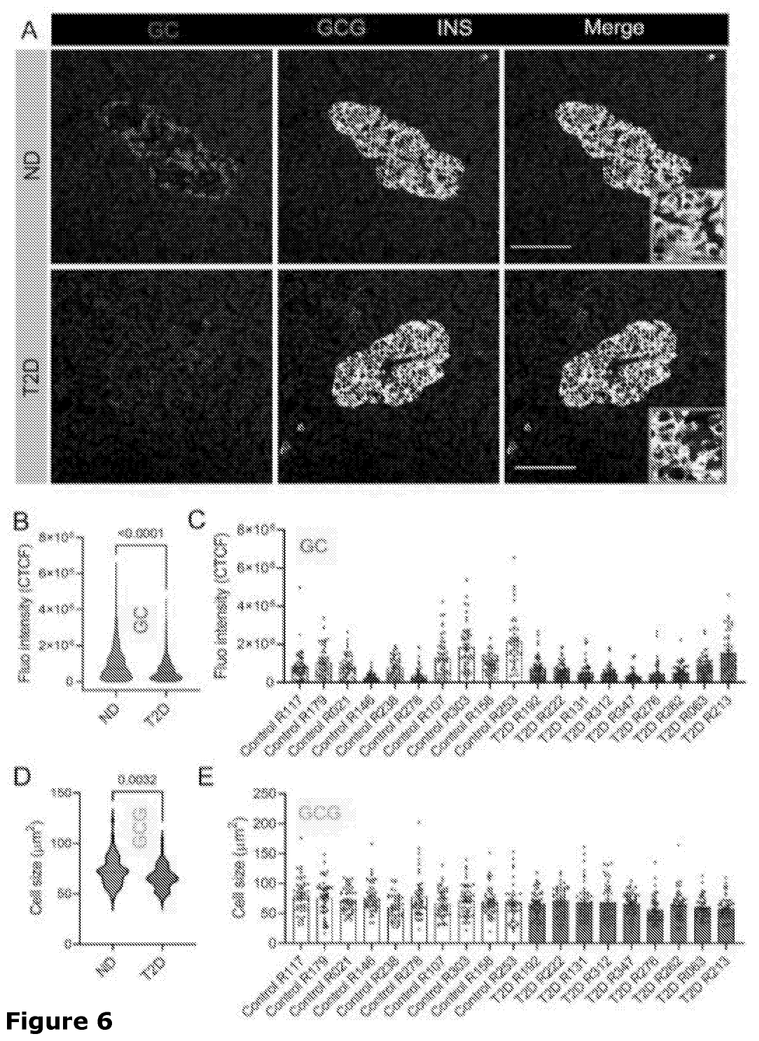

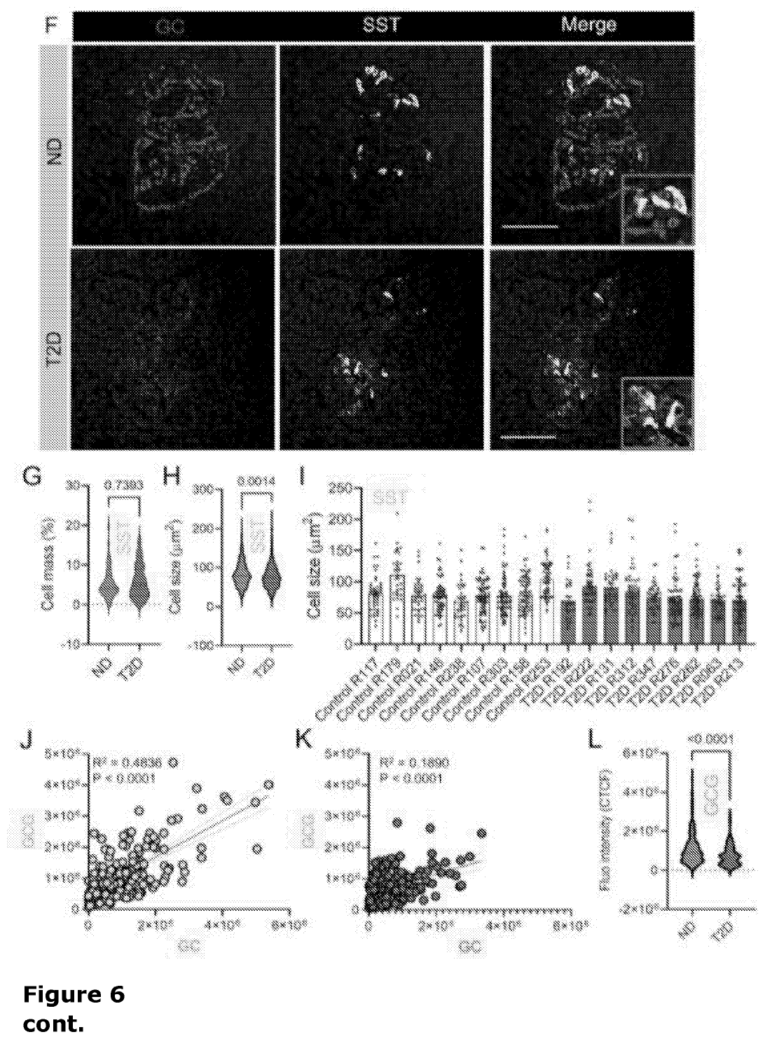

FIG. 6 illustrates GC and glucagon expression in islets from donors with and without type 2 diabetes. FIG. 6A) Representative images from non-diabetic (ND) and type 2 diabetes (T2D) donors showing a large reduction in GC expression, as well as decrease in α cell size. FIGS. 6B and 6C) Quantification using corrected total cell fluorescence (CTCF) reveals a highly significant decrease in GC expression in T2D versus ND donors (B), which is consistent across individual donors (C) (ND, n=89 cells from 10 donors; T2D, n=82 cells from and 9 donors) (Mann-Whitney test). FIGS. 6D and 6E) a cell size is significantly decreased in T2D versus ND donors (D), again consistent across donors (E) (ND, n=495 cells from 10 donors; T2D, n=450 cells from and 9 donors) (Mann-Whitney test). FIG. 6F) Representative images from non-diabetic (ND) and type 2 diabetes (T2D) donors showing a large reduction in GC expression, as well as decrease in δ cell size. FIGS. 6G-I) δ cell mass (G) is similar in ND and T2D donors, whereas δ cell size is reduced during T2D (H), consistent across individual donors (I) (ND, n=174 islets from 9 donors; T2D, n=210 islets from and 9 donors). FIGS. 6J and 6K) GC and GCG expression are strongly correlated in α cells of ND (J), but not T2D donors (K) (linear regression). FIG. 6L) GCG expression is significantly decreased in T2D versus ND donors (ND, n=200 cells from 10 donors; T2D, n=180 cells from and 9 donors). Scale bar=85 μm. GC, GC-globulin, GCG, glucagon, INS, insulin, SST, somatostatin.

### EXAMPLES

### Materials and Methods

### Experimental Design

No data were excluded unless the cells failed to respond to positive control, responded inappropriately to negative control, or displayed impaired viability. All individual data points are reported. The measurement unit is animal or batch of islets or pancreas section, with experiments replicated independently. Animals and islets were randomly allocated to treatment groups to ensure that all states were represented in the different experiment arms. Animals and pancreas sections were coded to allow blinded analysis.

### Mouse Models

GC^{-/-} mice harboring deletion of exon 5 of the GC gene were backcrossed to C57BL/6J for 10 generations⁹. These mice have undetectable circulating GC, as well as 25(OH)[3H]D3 binding⁹⁻¹⁰. Mice were housed in a specific pathogen-free facility with ad lib access to food and water. Vitamin D sufficiency was ensured by using chow supplemented with 1000 U/kg cholecalciferol. Mice were fed standard chow (SC) or high fat diet (HFD) containing 60% fat (Research Diets, Catalog # D12492), and body weight checked weekly from 0 to 12 weeks. Male and female mice were placed on SC or HFD from 8 weeks of age (numbers reported in the figure legends).

### Human Donors

Formalin-fixed paraffin-embedded pancreas sections were obtained from the Alberta Diabetes Institute IsletCore. Quality control and phenotyping data is available for each preparation via www.isletcore.ca.

### Glucose and Insulin Tolerance Tests

Mice were fasted for 4-5 hours before intraperitoneal injection of 1.5 g/kg of sterile filtered D-glucose. Tail vein sampling was performed at 0, 15, 30, 60, 90 and 120 mins post injection. Glucose levels were measured using a Contour XT glucometer (Bayer). For plasma glucagon measures, mice were fasted for 4-5 hours before intraperitoneal injection of 0.75 U/kg of insulin. Blood was collected at 0 and 30 mins post injection and stored at −80° C. pending ELISA for serum glucagon (Mercodia, Catalog #10-1281-01). Values lower than the assay detection limit were interpolated from the standard.

### Islet Isolation and Culture

Mice were humanely euthanized by rising CO₂ and cervical dislocation, before bile duct injection and inflation of the pancreas with 1 mg/ml SERVA NB8 collagenase (Amsbio, Catalog #17456.01). Islets were purified using Ficoll-Paque or Histopaque gradient separation and maintained at 37° C. and 5% CO2 in RPMI medium containing 10% FCS, 100 units/mL penicillin, and 100 μg/mL streptomycin.

### Immunostaining of Mouse Pancreases

Pancreases harvested before overnight incubation with 10% formalin overnight and dehydration and wax embedding. Sections were cut at 5 μm using a Leica microtome, before de-paraffinization and blocking with PBS-T+1% BSA for 1 hour. Sections were incubated with primary antibodies overnight at 4° C., before washing in PBS-T+0.1% BSA and incubation with secondary antibodies for 2 hours at room temperature. Primary antibodies used were: rabbit anti-insulin 1:500 (Cell Signaling Technology Cat #3014, RRID:AB_2126503); mouse monoclonal anti-glucagon 1:2000 (Sigma-Aldrich Cat #G2654, RRID:AB_259852); mouse anti-somatostatin 1:1000 (Thermo Fisher Scientific Cat #14-9751-80, RRID:AB_2572981) and rabbit anti-DBP 1:1000 (Sigma-Aldrich Cat #HPA019855, RRID:AB_1849545). Secondary antibodies were: goat anti-rabbit Alexa Fluor 633 (Thermo Fisher Scientific Cat #A-21052, RRID:AB 2535719), goat anti-rabbit Alexa Fluor 488 (Thermo Fisher Scientific Cat #R37116, RRID:AB_2556544), goat anti-guinea pig Alexa Fluor 488 (Thermo Fisher Scientific Cat #A-11073, RRID:AB_2534117), goat anti-mouse Alexa Fluor 488 (Thermo Fisher Scientific Cat #A-11029, RRID:AB_138404) and goat anti-guinea pig Alexa Fluor 568 (Thermo Fisher Scientific Cat #A-11075, RRID:AB_2534119), applied at 1:1000. Specificity of antibodies was based upon known cell type co-localizations, overlap with insulin, glucagon or somatostatin reporters, or loss of staining in knockout tissue. F-actin and G-actin were visualized using Phalloidin-488 (Abcam Cat #ab176753) or DNaseI-594 (Invitrogen Cat #D12372), respectively.

Imaging was performed using either Zeiss LSM780 or LSM880 meta-confocal microscopes, equipped with sensitive GaAsP spectral detectors. Alexa Fluor 488, Alexa Fluor 568 and Alexa Fluor 633 were excited at λ=488 nm, λ=568 and λ=633 nm, respectively. Emitted signals for Alexa Fluor 488, Alexa Fluor 568 and Alexa Fluor 633 were detected at λ=498-559, nm λ=568-629 and λ=633-735 nm, respectively. Super-resolution images of F-actin were acquired using a Nikon N-SIM S microscope, SR HP Apo TIRF 100×1.49 NA/oil immersion objective and ORCA-Flash 4.0 sCMOS camera, with online deconvolution. Alexa Fluor 488 was excited at λ=500-550 nm, with emitted signals detected at λ=570-640 nm.

### Intracellular Ca₂₊ Imaging

The ratiometric Ca²⁺ dye, Fura2 (HelloBio, Catalog #HB0780-1mg), was loaded into islets using 20% pluronic acid dissolved in DMSO. Islets were transferred to the heated chamber (34 C) of a Nikon Ti-E microscope coupled to a 10×/0.3 NA air objective (Nikon Plan Fluor), allowing simultaneous cell resolution imaging of multiple islets (lateral resolution=910 nm). A Cairn Research FuraLED system provided excitation at λ=340 nm and λ=385 nm. Emitted signals were captured at λ=470-550 nm using a Photometric Delta Evolve EM-CCD. Intracellular Ca²⁺ levels were shown as the emission ratio at 340 nm and 385 nm. A number of experiments were repeated using the non-ratiometric Ca²⁺ dye Fluo-8 (AAT Bioquest, Catalog #20494). Confocal excitation was delivered at 470 nm (emission λ=500-550 nm) by a North 89 LDI Illuminator, CrestOptics V2 X-light spinning disk and 20×/0.8 NA air objective. Intracellular Ca²⁺ levels were quantified as F/Fₘᵢₙ where F=fluorescence at any given timepoint, and Fₘᵢₙ=mean minimum fluorescence. All experiments were performed in HEPES-bicarbonate buffer was used, containing (in mmol/L) 120 NaCl, 4.8 KCl, 24 NaHCO3, 0.5 Na2HPO4, 5 HEPES, 2.5 CaCl2, 1.2 MgCl2, and supplemented with 0.5-17 mM D-glucose.

### Insulin and Glucagon Secretion Assays

HEPES-bicarbonate buffer was used for all assays, containing (in mmol/L) 120 NaCl, 4.8 KCl, 24 NaHCO3, 0.5 Na2HPO4, 5 HEPES, 2.5 CaCl2, 1.2 MgCl2+0.1% BSA. For glucagon secretion, batches of 10 islets were pre-incubated in buffer supplemented with 10 mM glucose, before incubation with either 10 mM glucose, 0.5 mM glucose or 0.5 mM glucose+5 μM epinephrine for 1 hr at 37° C. Glucagon released into the supernatant was then measured using either HTRF ultrasensitive assay (Perkin-Elmer, Catalog #62CGLPEG) or Lumit bioluminescent immunoassay (Promega)¹¹. For insulin secretion, batches of 10 islets were pre-incubated in buffer supplemented with 1 mM glucose, before sequential incubation in 3 mM glucose, 17 mM glucose and 17 mM glucose+10 mM KCI for 30 minutes at 37° C. Insulin was measured using HTRF ultrasensitive assay (Perkin-Elmer, Catalog #62IN2PEG) or Lumit bioluminescent immunoassay (Promega). Total glucagon and insulin were extracted from islets lysed in acid ethanol.

### Image Analysis

Ca²⁺ imaging: α cells were identified in an unbiased manner by their characteristic Ca²⁺ spiking activity at low glucose, as well as responses to epinephrine, as reported^{10, 12} Signal contributions from β cells are unlikely given that they are electrically silent at low glucose and inhibited by epinephrine. The proportion of low glucose-responsive α cells was calculated as the area occupied by identified α cells normalized to total islet area. Ca²⁺ spike amplitude was calculated for individual cells using delta 340/385 nm or F/Fₘᵢₙ.

Immunostaining: GC, F-actin, G-actin and glucagon were analyzed using corrected total cell fluorescence (CTCF), as previously described. CTCF accounts for the effect of cell size on fluorophore intensity by taking the integrated density and subtracting area of the selected cell x mean background fluorescence^{13,14} α cell, β cel and δ cell area and size were analysed using ImageJ (NIH) Particle Analysis plugin applied to binarized and thresholded images. Linear adjustments to brightness and contrast were applied to representative images, with intensity values maintained between samples to allow cross-comparison.

### Statistics

Statistical details for each experiment are reported in the corresponding figure legend. The n number represents animal, batch of islets or donor. No data were excluded unless the cells displayed a clear non-physiological state (i.e. impaired viability), and all individual data points are reported in the figures. Data normality was assessed using D'Agostino-Pearson test. All analyses were conducted using GraphPad Prism 9.0 software. Pairwise comparisons were made using two-tailed unpaired t test (parametric) or Mann-Whitney test (non-parametric). To assess multiple interactions, one-way or two-way ANOVA were used, adjusted for repeated measures where needed. Post hoc comparisons were made using Bonferonni's, accounting for degrees of freedom. Linear regression was used to assess strength of association between explanatory and dependent variables, with slopes compared using analysis of covariance. Data represent mean±SEM or SD, with individual datapoints shown where possible. Where a large number of datapoints obscure mean±SEM or SD, violin plots are instead used (showing median and interquartile range).

### Results

### Deletion of GC Increases Basal Glucagon Secretion During High Fat Diet

Mice with global GC deletion were used, since: 1) GC is exclusively expressed in a cells and liver^{6, 10}; 2) Gcg-Cre lines have variable recombination efficiency and specificity¹⁵; 3) recently reported Gog-CreERT knock-in mice require tamoxifen induction¹⁶, which interferes with hepatic triglyceride accumulation and hence GC levels; and 4) two patients with homozygous inactivating mutations in GC have been described¹⁷⁻¹⁸. The GC^{-/-} mice used here are phenotypically well-validated, do not possess detectable GC/Gc expression, and have 50% reduced and 90% reduced 25(OH)D and 1,25(OH)D levels, respectively^{9, 10}.

GC^{-/-} and littermate control GC^{+/+} mice were placed on high fat diet (HFD) for up to 12 weeks, with glucose tolerance tested every 4 weeks. Glucose tolerance was similar in GC^{+/+} and GC^{-/-} mice under standard diet (i.e. 0 weeks HFD) (FIG. 1A and B). No significant differences were observed in glucose tolerance in GC^{+/+} and GC^{-/-} mice at 4 weeks, 8 weeks and 12 weeks HFD (FIG. 1C-H). Confirming efficacy of the preclinical obesity model, 4-week HFD-fed GC^{+/+} and GC^{-/-} mice were glucose intolerant versus age-matched controls fed standard chow (FIG. 1I). Body weight gain was similar in female and male GC^{+/+} and GC^{-/-} mice during HFD (FIG. 1J and K).

As a similar phenotype was observed in both female and male mice, both sexes were combined for subsequent studies. Plasma glucagon levels were assessed at 0 and 30 mins post-injection of insulin. While glucose levels were lowered to a similar extent in GC^{+/+} and GC^{-/-} mice (FIG. 1L), basal fasted glucagon secretion was significantly (2-fold) elevated in GC^{-/-} mice after 4 weeks of HFD (FIG. 1M-O). Glucagon: glucose ratios, calculated using measures from the same animal, provided further evidence of dysregulated basal but not stimulated glucagon secretion (FIG. 1M and P).

In summary, GC^{-/-} mice are glucose tolerant during HFD, but display elevated basal glucagon levels, indicative of dysregulated α cell function.

HFD GC^{-/-} Mice Have Aberrant α-, β-and 8-Cell Morphology

Pancreata isolated from HFD-fed GC^{+/+} mice showed a 2-fold increase in GC protein levels versus standard chow (SC) controls (FIG. 2A and B). GC protein was undetectable in pancreata from HFD-fed GC^{-/-} mice, further demonstrating the reliability of the antibody and immunostaining approach used10 (FIG. 2A and B). It has previously been shown that pancreata from SC-fed GC^{-/-} mice possess decreased α cell mass and α cell size¹⁰. High resolution morphometric analysis in pancreata from HFD-fed mice was thus performed.

HFD feeding itself did not affect islet area occupied by α cells, nor α cell size, as compared to age-matched SC controls (FIG. 2C-E). However, a large reduction in a cell size was observed in HFD-fed GC^{-/-} mice versus GC^{+/+} littermates (FIG. 2C-E). By contrast to its effects on α cells, HFD increased β cel size in GC_{+/+}, with a further increase detected in GC^{-/-} islets (FIG. 2C, F and G). Analysis of δ cells revealed a HFD-induced increase in their proportion, a change that was partly reversed by deletion of GC (FIG. 2H-J).

In summary, these data suggest that, during HFD, GC restrains β cell size, while promoting α cell size and δ cell expansion to support normal plasticity.

Glucagon Secretion and α cell Ca²⁺ Responses are Impaired in HFD GC^{-/-} Islets

Islets were isolated from HFD-fed GC^{+/+} and GC^{-/-} mice and their age-matched standard chow controls for detailed in vitro analyses. The SC diet cohort included some heterozygous (GC^{+/-}) animals as control, since we did not see a difference compared to wild-types (GC^{+/+}). As reported previously¹⁰, SC GC^{-/-} islets presented with impaired low glucose-and low glucose+epinephrine-stimulated glucagon secretion versus GC^{+/+} littermates (FIG. 3A). Similar impairments were detected for HFD, although responses to epinephrine remained intact, suggesting that the defect is upstream of the exocytotic machinery (FIG. 3A). At glucose concentration sub-maximal for alpha cell function (i.e. 2 mM), glucagon secretion still tended to be reduced in HFD GC^{-/-} islets (FIG. 3B). Glucose-stimulated insulin secretion tended to be increased in SC GC^{-/-} islets and this trend became significant during HFD (FIG. 3C). A tendency toward increased basal glucagon secretion was also noted in HFD GC^{-/-} islets (FIG. 3B), which might partly explain the increased insulin secretion, since glucagon is insulinotropic when beta cells are active¹⁹. No significant differences in total glucagon or insulin content could be detected between GC^{-/-} islets and GC^{+/+} controls (FIG. 3 D and E).

Given the apparent changes in glucagon and insulin secretion, we next investigated upstream Ca²⁺ fluxes, with α cells identified by their characteristic responses to low glucose (0.5 mM) as well as epinephrine¹². Confirming our previous findings, proportion active α cells (i.e. % cells displaying Ca²⁺ spikes; a measure of recruitment into Ca²⁺ activity) was decreased in SC GC^{-/-} islets (FIG. 3F-I). By contrast to previous results, a significant decrease in Ca²⁺ amplitude in SC GC^{-/-} islets was also observed (FIG. 3G-I). The most likely explanation for this discrepancy is the relatively advanced age of the SC mice used in the study here, which were age-matched with those receiving HFD, and suggests that age might exacerbate the in vitro phenotype following GC deletion. Nonetheless, HFD decreased both the proportion of active α cells, as well as the amplitude of their Ca²⁺ spikes (FIG. 3F-I). The effect of HFD on Ca²⁺ spike amplitude, but not proportion active α cells, was exacerbated following loss of GC (FIG. 3F-I). Ca²⁺ imaging results were validated using a second Ca²⁺ probe (Fluo8), confocal microscopy and a higher magnification objective (FIG. 3J-K).

### GC-Dependent Actin Cytoskeleton Remodelling Occurs During HFD

During stimulation, the F-actin cytoskeleton undergoes rearrangement to facilitate exocytosis of hormone vesicles²⁰⁻²². In line with the actin-scavenging function of GC, it has previously been shown that F-actin density was increased in GC^{-/-} islets, leading to changes in glucagon granule morphology and distribution, suggestive of sequestration and trapping¹⁰. Directly implicating the F-actin cytoskeleton in glucagon release, incubation of GC^{-/-} islets with Latrunculin B was able to restore function¹⁰. We therefore investigated whether restoration of GC level and ergo F-actin cytoskeletal structure might rescue the phenotype of HFD GC^{-/-} islets. Following acute (10 mins) stimulation with low glucose, F-actin density was decreased in GC^{-/-} islets but unchanged in islets from GC^{+/+} islets (FIG. 4A-C). F-actin remained low in GC^{-/-} islets after chronic (60 mins) stimulation, but was increased ˜2-fold in GC^{+/+} islets (FIG. 4A-C).

As shown previously¹⁰, deletion of GC from standard chow islets led to increased F-actin density, concomitant with a decrease in G-actin monomers, presumably due to their involvement in forming polymerized actin (FIG. 4D-F). On the other hand, F-actin density and fiber thickness increased by almost 3-fold in HFD GC^{+/+} islets (FIG. 4D-F). Unexpectedly, given its actin scavenging function, deletion of GC led to a decrease in F-actin density in HFD GC^{-/-} islets versus GC^{+/+} controls (FIG. 4D-E). By contrast, monomeric G-actin was increased in HFD GC^{-/-} islets, suggesting that G-actin is sequestered away from sites of F-actin polymerization following deletion of GC (FIG. 4D-F). In all cases, changes in F-actin and G-actin were detected throughout the islet (FIG. 4E and F) as well as in individual α cells (FIG. 4G and H) and β cells (FIG. 4I and J), suggesting that GCG granule-resident GC acts in a paracrine manner to influence cytoskeletal structure throughout the islet i.e. by severing and depolymerizing F-actin into G-actin^{10, 22}.

### GC Supplementation Restores F-Actin Cytoskeletal Structure and Glucagon Release, as Well as Amplifies Insulin Secretion

We next investigated whether exogenous GC could modify F-actin levels in GC^{-/-} islets to restore α cell activity. Using a published RNA-seq dataset⁷, transcripts for the endocytic receptors responsible for GC uptake, megalin (Lrp2) and cubilin (Cubn) (14; 39-41), were found to be expressed in purified α cells at a similar level to the gastric inhibitory polypeptide receptor (Gipr) (normalized expression: 8.9±5.3 versus 9.7±3.4 versus 8.0±7.0 for Lrp2 versus Cubn versus Gipr, respectively) (taken from (GSE76017). As expected from this, GC levels could be restored in HFD GC^{-/-} islets following incubation with exogenous protein (FIG. 5A-C). Confirming the directionality of F-actin changes, treatment of HFD GC^{-/-} islets with GC restored F-actin levels to wild-type levels (FIG. 5D), as seen throughout the islet as well as in individual α cells (FIG. 5C-E).

As expected, low glucose (G0.5)-stimulated glucagon secretion was impaired in HFD GC^{-/-} islets¹⁰. Pertinently, application of GC restored normal glucagon secretion in HFD GC^{-/-} islets, without influencing the function of HFD GC^{+/+} islets (FIG. 5F). The effects of GC on glucagon secretion were not associated with increases in intracellular Ca²⁺ concentration, which was slightly but significantly decreased in GC-treated islets (FIG. 5G and H). Reflecting either the lowered glucagon tone or decreased F-actin in GC^{-/-} islets, insulin secretion failed to shut off at low glucose (0.5 mM) (FIG. 5I), an effect remarkably similar to that reported when the small GTPase and actin polymerizer, RhoA, is inhibited in α cells. GC treatment was unable to restore this defect or influence basal insulin levels in either GC^{+/+} or GC^{-/-} islets (FIG. 5I). By contrast, GC treatment led to a large (˜10-fold) amplification of glucose-stimulated insulin secretion, with a greater effect in HFD GC^{-/-} islets (FIG. 5J).

Finally, as a proof of principle, it was shown that GC could be supplemented in human islets, leading to increases in glucagon granule area as well as F-actin density (FIG. 5K-M), visualized at ˜110 nm resolution using structured illumination microscopy.

### GC Expression is Decreased in Islets of T2D Donors

In pancreas sections from non-diabetic (ND) donors, GC expression was only present in α cells, as expected^{6, 10, 23} (FIG. 6A). While a similar staining pattern was observed in pancreas sections from T2D donors, GC expression levels were markedly (˜2-fold) reduced (FIG. 6A). Some inter-individual variability was observed, but reduced GC expression appeared to be a remarkably consistent feature of T2D (FIG. 6B and C). Reflecting findings in HFD mice, analysis of individual α cells in T2D donors revealed a decrease in cell size (FIG. 6D and E). While proportion of islet area occupied by δ cells was unchanged during T2D, δ cell size was slightly but significantly reduced (FIG. 6F-I).

Linear regression showed a strong correlation between GC and GCG (glucagon) expression in α cells from donors without diabetes (FIG. 6J). Whilst a significant linear correlation was also detected for individuals with T2D, the strength of correlation was much lower (FIG. 6K), consistent with the reported decrease in GC expression (FIG. 6B), as well α cell glucagon expression (FIG. 6L). As expected from this, the regression slopes were significantly different between ND and T2D samples (P<0.001). Together, these analyses show that glucagon expression co-varies with GC expression and that this relationship is partly lost during T2D.

### Discussion

It has been shown that deletion of GC in HFD-fed animals leads to basal hyperglucagonemia and impaired low glucose-stimulated glucagon secretion. These secretory defects are associated with changes in Ca²⁺ fluxes, α cell, β cell and δ cell size and mass, as well as F-actin and G-actin abundance. α cell function can be restored in GC^{-/-} islets by using exogenous GC, which is taken up into cells following culture. Lastly, islets from donors with T2D show decreases in GC expression, with concomitant changes in α cell and δ cell size and mass. Together, these results expand previous findings on GC by revealing its regulatory role in glucagon secretion during metabolic stress, and further suggest that GC is a pivotal component of the α cell phenotype in health and disease. While GC is a signature gene expressed in α cells, the current study shows that α cells also have the potential to acquire GC via megalin-mediated endocytosis. This raises the possibility that circulating levels of GC may also contribute to α cell GC-actin dynamics and phenotype.

In vivo metabolic phenotyping demonstrated that GC^{+/+} and GC^{-/-} mice possess similar glucose excursion curves in response to intraperitoneal glucose injection. However, basal plasma glucagon concentrations were consistently raised in GC^{-/-} mice, in line with a tendency toward elevated glucagon secretion from isolated islets at 17 mM glucose, which would be expected to increase hepatic glucose output. One possible explanation for the apparently normal glucose homeostasis is that the increase in glucagon levels is not sufficient to influence insulin counter-regulation, or might even act to prime β cels for insulin secretion^{24, 25}. Alternatively, recent studies have shown that Gc is upregulated in de-differentiated β cells and deletion of GC increases glucose-stimulated insulin secretion and liver insulin sensitivity at 12 weeks HFD²⁶. In any case, these data show that HFD-induced basal hyperglucagonemia²⁷ is further aggravated following GC deletion. It was not possible to reliably detect significant increases in in vitro insulin secretion or β cell function at 4-8 weeks HFD, arguing against this possibility here, although it is conceded that clamp studies are needed to properly assess this. Moreover, GC expression was variably upregulated in β cells, remaining much lower than the levels seen in α cells.

Plasma glucagon levels, stimulated by insulin injection, were almost identical in HFD-fed GC^{+/+} and GC^{-/-} mice, despite impaired glucagon release from isolated islets incubated in low (0.5 mM) glucose. It was found, however, that the effect of GC deletion was milder in islets exposed to sub-maximal (2 mM) glucose concentration, which would be closer to that achieved in vivo. Together, these data suggest that GC is relatively more important in alpha cells operating close to their functional ceiling, with the caveat that in vitro glucagon secretion assays might be less sensitive at 2 mM glucose due to the relatively smaller magnitude change. Along similar lines, HFD-fed GC^{-/-} mice presented with basal hyperglucagonemia at blood glucose concentrations ˜10-11 mmol/L, whilst in isolated islets basal glucagon secretion was similar in GC^{-/-} and GC^{+/+} mice at high glucose. One explanation for this discrepancy might lie in the finding that glucose-stimulated insulin secretion was almost 2-fold increased in HFD-fed GC^{-/-} islets. In vivo, relative hyperinsulinemia would be expected to drive hyperglucagonemia to maintain blood glucose levels, which were not different between HFD-fed GC^{+/+} and GC^{-/-} mice. Another explanation might lie in the changes in a cell morphology observed in pancreas sections taken from HFD-fed GC^{-/-} animals. A decrease in α cell size might lead to an increase in α cell membrane juxtaposed with the islet capillaries, favoring release of glucagon into the circulation.

During standard diet, it was shown that loss of GC leads to a large increase in the density of the F-actin cytoskeleton (and concomitant decrease in G-actin), acting as a physical barrier against exocytosis of glucagon granules during low glucose stimulation¹⁰. Changes in the F-actin and G-actin cytoskeleton occur throughout the islet, since ˜50% of GC is present in glucagon granules and can readily be taken up by neighbouring cells by endocytosis, as shown here following application of exogenous GC. Following 4-8 weeks HFD, F-actin density was increased almost 2-fold in GC^{+/+} islets. On a background of metabolic stress, deletion of GC did not further increase F-actin density. In fact, HFD GC^{-/-} islets showed a surprising reduction in F-actin density, contrary to our previous findings in standard diet islets¹⁰. A similar decrease in F-actin density was seen in GC^{-/-} islets stimulated with low glucose for 60 mins. Notably, treatment with exogenous GC replenished intracellular GC and F-actin levels in HFD GC^{-/-} islets, confirming that GC acts to increase F-actin density during metabolic stress. Given that GC is a potent actin scavenger, what might be the mechanisms involved in this apparent decrease in F-actin? A likely mechanism revolves around G-actin, which was virtually undetectable in HFD GC^{-/-} islets. Without G-actin to supply monomers, polymerized F-actin cannot be formed. Indeed, previous reports have shown a similar decrease in F-actin in trophoblasts depleted for GC, which was associated with an increase in G-actin monomers in the nucleus where they are unavailable for assembly into polymerized F-actin28. Another mechanism might be a large compensatory upregulation in gelsolin, which severs F-actin into G-actin^{20,29}, although we would expect this to be associated with an increase in G-actin levels.

Kuo et al. recently reported that GC^{-/-} islets show an insulin signaling/sensitivity defect, but exhibit a normal glucagon phenotype under both standard diet and high fat diet conditions²⁶. Since studies that used islets from animals on 4 and 8 week HFD, it cannot be excluded that glucagon secretion in GC^{-/-} animals/islets normalizes in line with improved β cel function at 12 weeks, the feeding period used by Kuo et al. A different GC knockout mouse line was also used, which might give rise to a different phenotype. However, it should be noted that these animals are well-validated by multiple groups and show complete loss of GC in α cells and the liver, undetectable circulating GC based upon LC-MS, and a 90% reduction in circulating 25(OH)D in homozygotes⁹⁻¹⁰. Suggesting that GC plays a critical role in α cell biology: 1) GC is an a cell signature gene; 2) GC protein expression is upregulated during HFD, remaining 10-100-fold higher than that in β cels; and 3) defects in α cell function have a clear mechanistic basis, including changes in cell morphology, cell mass, cytoskeletal structure and ionic fluxes, shown also by recent Patch-seq studies³⁰. Nonetheless, these studies together posit that, depending on duration of metabolic stress, effects of GC deletion (and GC supplementation) can be seen on both the insulin and glucagon axes. Further studies using conditional GC deletion in α cells and β cells are warranted.

Studies in human donor pancreas sections revealed that GC and glucagon expression are positively associated, with levels co-varying across hundreds of individual cells examined, a relationship that was lost during T2D. Mechanistically, this observation likely reflects changes in the alpha cell transcription factor network, since GC possesses cell type-selective open chromatin regions⁶. Ultimately, however, altered gene regulation must impact functional protein targets, and our in vitro findings support the notion that the disrupted relationship between GC and glucagon might contribute to impaired glucagon secretion during T2D. Further studies are warranted in isolated human islets to investigate the effects of silencing GC on glucagon expression and secretion in α cells.

Changes in the actin cytoskeleton could be rescued using exogenous GC. In the kidney, GC-bound 25(OH)D is taken up by facilitated endocytotic uptake via the megalin-cubulin complex^{2, 23, 31}, where liberated 25(OH)D is then converted to 1,25(OH)2D. Immunostaining clearly showed dose-dependent uptake of GC into islets, demonstrating that similar transport mechanisms also exist in the pancreas, as suggested by published RNA-seq data³². These data suggest that, unusually, decreases in expression of a key cell signature gene can be offset by supplementing its protein product and warrant further investigation of the uptake mechanisms involved. While these results point to GC as a therapeutic target, caution should be extended due to opposing effects of GC on both the α cell and β cel compartments²⁶. However, it should be noted that high glucose levels have been shown to inhibit megalin-455 mediated endocytosis, which might differentially affect GC uptake into α cells and β cells³³. Moreover, molecular addresses such as VIBR could be used to target GC specifically to α cells³⁴⁻³⁶. In any case, we envisage that GC administration during type 2 diabetes could maintain α cell function, whilst restraining β cell proliferation and hyperinsulinemia, known to drive insulin resistance³⁷⁻³⁸. Data in human pancreas sections supports a reduction in GC during type 2 diabetes, lending further weight to this argument. Nonetheless, careful preclinical studies in mice at various timepoints are required to assess this.

In summary, it is shown that α cells lacking GC fail to adapt properly to metabolic stress, displaying a range of defects leading to impaired basal and low glucose-stimulated glucagon release. Given its role under both normal and obesogenic conditions, GC should thus be considered as a key regulator of α cell function and glucagon secretion.

### REFERENCES

1. Daiger S P, Schanfield M S, Cavalli-Sforza L L: Group-specific component (Gc) proteins bind vitamin D and 25-hydroxyvitamin D. Proc Natl Acad Sci USA 1975; 72:2076-2080
2. Bouillon R, Schuit F, Antonio L, Rastinejad F: Vitamin D Binding Protein: A Historic Overview. Frontiers in Endocrinology 2020; 10
3. Mc Leod J F, Kowalski M A, Haddad J G, Jr.: Interactions among serum vitamin D binding protein, monomeric actin, profilin, and profilactin. J Biol Chem 1989; 264:1260-1267
4. Harper K D, McLeod J F, Kowalski M A, Haddad J G: Vitamin D binding protein sequesters monomeric actin in the circulation of the rat. Journal of Clinical Investigation 1987; 79:1365-1370
5. Bikle Daniel D: Vitamin D Metabolism, Mechanism of Action, and Clinical Applications. Chemistry & Biology 2014; 21:319-329
6. Ackermann A M, Wang Z, Schug J, Naji A, Kaestner K H: Integration of ATAC-seq and RNA-seq identifies human alpha cell and beta cell signature genes. Mol Metab 2016; 5:233-244
7. Adriaenssens A E, Svendsen B, Lam B Y H, Yeo G S H, Holst J J, Reimann F, Gribble F M: Transcriptomic profiling of pancreatic alpha, beta and delta cell populations identifies delta cells as a principal target for ghrelin in mouse islets. Diabetologia 2016; 59:2156-2165
8. Akerman I, Tu Z, Beucher A, Rolando D M Y, Sauty-Colace C, Benazra M, Nakic N, Yang J, Wang H, Pasquali L, Moran I, Garcia-Hurtado J, Castro N, Gonzalez-Franco R, Stewart A F, Bonner C, Piemonti L, Berney T, Groop L, Kerr-Conte J, Pattou F, Argmann C, Schadt E, Ravassard P, Ferrer J: Human Pancreatic beta Cell IncRNAs Control Cell-Specific Regulatory 568 Networks. Cell Metab 2017; 25:400-411
9. Safadi F F, Thornton P, Magiera H, Hollis B W, Gentile M, Haddad J G, Liebhaber S A, Cooke N E: Osteopathy and resistance to vitamin D toxicity in mice null for vitamin D binding protein. J Clin Invest 1999; 103:239-251
10. Viloria K, Nasteska D, Briant L J B, Heising S, Larner D P, Fine N H F, Ashford F B, da Silva Xavier G, Ramos M J, Hasib A, Cuozzo F, Manning Fox J E, MacDonald P E, Akerman I, Lavery G G, Flaxman C, Morgan NG, Richardson S J, Hewison M, Hodson D J: Vitamin-D-Binding Protein Contributes to the Maintenance of α Cell Function and Glucagon Secretion. Cell Reports 2020; 31
11. El K, Gray S M, Capozzi M E, Knuth E R, Jin E, Svendsen B, Clifford A, Brown J L, Encisco S E, Chazotte B M, Sloop K W, Nunez D J, Merrins M J, D'Alessio D A, Campbell J E: GIP mediates the incretin effect and glucose tolerance by dual actions on α cells and β cells. Science Advances 2021; 7
12. Hamilton A, Zhang Q, Salehi A, Willems M, Knudsen J G, Ringgaard A K, Chapman C E, Gonzalez-Alvarez A, Surdo N C, Zaccolo M, Basco D, Johnson P R V, Ramracheya R, Rutter G A, Galione A, Rorsman P, Tarasov A I: Adrenaline Stimulates Glucagon Secretion by Tpc2-Dependent Ca2+Mobilization From Acidic Stores in Pancreatic α-Cells. Diabetes 2018; 67:1128-1139
13. Gavet O, Pines J: Progressive activation of CyclinB1-Cdk1 coordinates entry to mitosis. Dev Cell 2010; 18:533-543
14. McCloy R A, Rogers S, Caldon C E, Lorca T, Castro A, Burgess A: Partial inhibition of Cdk1 in G 2 phase overrides the SAC and decouples mitotic events. Cell Cycle 2014; 13:1400-1412
15. da Silva Xavier G, Hodson D J: Mouse models of peripheral metabolic disease. Best Practice & Research Clinical Endocrinology & Metabolism 2018
16. Ackermann A M, Zhang J, Heller A, Briker A, Kaestner K H: High-fidelity Glucagon-CreER Mouse line generated by CRISPR-Cas9 assisted gene targeting. Mol Metab 2017; 6:236-244
17. Henderson C M, Fink S L, Bassyouni H, Argiropoulos B, Brown L, Laha T J, Jackson K J, Lewkonia R, Ferreira P, Hoofnagle A N, Marcadier J L: Vitamin D-Binding Protein Deficiency and Homozygous Deletion of the GC Gene. New England Journal of Medicine 2019; 380:1150-1157
18. Banerjee R R, Spence T, Frank S J, Pandian R, Hoofnagle A N, Argiropoulos B, Marcadier J L: Very Low Vitamin D in a Patient With a Novel Pathogenic Variant in the GC Gene That Encodes Vitamin D-Binding Protein. J Endocr Soc 2021; 5:bvab104
19. Capozzi M E, Wait J B, Koech J, Gordon A N, Coch R W, Svendsen B, Finan B, D'Alessio D A, Campbell J E: Glucagon lowers glycemia when beta-cells are active. JCI Insight 2019; 5
20. Tomas A, Yermen B, Min L, Pessin J E, Halban P A: Regulation of pancreatic beta-cell insulin secretion by actin cytoskeleton remodelling: role of gelsolin and cooperation with the MAPK signalling pathway. J Cell Sci 2006; 119:2156-2167
21. Hoboth P, Muller A, Ivanova A, Mziaut H, Dehghany J, Sonmez A, Lachnit M, Meyer-Hermann M, Kalaidzidis Y, Solimena M: Aged insulin granules display reduced microtubule-dependent mobility and are disposed within actin-positive multigranular bodies. Proc Natl Acad Sci USA 2015; 112:E667-676
22. Kalwat M A, Thurmond D C: Signaling mechanisms of glucose-induced F-actin remodeling in pancreatic islet β cells. Experimental & Molecular Medicine 2013; 45:e37-e37
23. Lam C J, Chatterjee A, Shen E, Cox A R, Kushner J A: Low-Level Insulin Content Within Abundant Non-β Islet Endocrine Cells in Long-standing Type 1 Diabetes. Diabetes 2019; 68:598-608
24. Capozzi M E, Svendsen B, Encisco S E, Lewandowski S L, Martin M D, Lin H, Jaffe J L, Coch R W, Haldeman J M, MacDonald P E, Merrins M J, D'Alessio D A, Campbell J E: beta Cell tone is defined by proglucagon peptides through cAMP signaling. JCI Insight 2019; 4
25. El K, Capozzi M E, Campbell J E: Repositioning the Alpha Cell in Postprandial Metabolism. Endocrinology 2020; 161
26. Kuo T, Damle M, González B J, Egli D, Lazar M A, Accili D: Induction of α cell-restricted Gc in dedifferentiating β cels contributes to stress-induced β cell dysfunction. JCI Insight 2019; 4
27. Kellard J A, Rorsman N J G, Hill T G, Armour S L, van der Bunt M, Rorsman P, Knudsen J G, Briant L J B: Reduced somatostatin signalling leads to hypersecretion of glucagon in mice fed a high fat diet. Molecular Metabolism 2020
28. Ganguly A, Tamblyn J A, Shattock A, Joseph A, Larner D P, Jenkinson C, Gupta J, Gross S R, Hewison M: Trophoblast uptake of DBP regulates intracellular actin and promotes matrix invasion. J Endocrinol 2021; 249:43-55
29. Nelson T Y, Boyd A E, 3rd: Gelsolin, a Ca2+-dependent actin-binding protein in a hamster insulin-secreting cell line. Journal of Clinical Investigation1985; 75:1015-1022
30. Dai X-Q, Camunas-Soler J, Briant L J B, Santos T d, Spigelman A F, Walker E M, Arrojo e Drigo R, Bautista A, Jones R C, Lyon J, Nie A, Smith N, Fox J E M, Kim S K, Rorsman P, Stein R W, Quake S R, MacDonald P E: Heterogenous impairment of α-cell function in type 2 diabetes is linked to cell maturation state. 2021
31. Nykjaer A, Dragun D, Walther D, Vorum H, Jacobsen C, Herz J, Melsen F, Christensen E I, Willnow T E: An Endocytic Pathway Essential for Renal Uptake and Activation of the Steroid 25-(OH) Vitamin D3. Cell 1999; 96:507-515
32. Blodgett D M, Nowosielska A, Afik S, Pechhold S, Cura A J, Kennedy N J, Kim S, Kucukural A, Davis R J, Kent S C, Greiner D L, Garber M G, Harlan D M, dilorio P: Novel Observations From Next-Generation RNA Sequencing of Highly Purified Human Adult and Fetal Islet Cell Subsets. Diabetes 2015; 64:3172-3181
33. Peruchetti D d B, Silva-Aguiar R P, Siqueira G M, Dias W B, Caruso-Neves C: High glucose reduces megalin-mediated albumin endocytosis in renal proximal tubule cells through protein kinase B O-GlcNAcylation. Journal of Biological Chemistry 2018; 293:11388-11400
34. Kim A, Knudsen J G, Madara J C, Benrick A, Hill T, Abdul-Kadir L, Kellard J A, Mellander L, Miranda C, Lin H, James T, Suba K, Spigelman A F, Wu Y, Macdonald P E, Wernstedt Asterholm I, Magnussen T, Christensen M, Visboll T, Salem V, K Knop F, Rorsman P, Lowell B B, Briant L J B: Arginine-vasopressin mediates counter-regulatory glucagon release and is diminished in type 1 diabetes. eLife 2021; 10
35. Liu L, Dattaroy D, Simpson K F, Barella L F, Cui Y, Xiong Y, Jin J, König G M, Kostenis E, 662 Roman J C, Kaestner K H, Doliba N M, Wess J: α-cell Gq signaling is critical for maintaining euglycemia. JCI Insight 2021
36. Ammala C, Drury W J, 3rd, Knerr L, Ahlstedt I, Stillemark-Billton P, Wennberg-Huldt C, Andersson E M, Valeur E, Jansson-Lofmark R, Janzen D, Sundstrom L, Meuller J, Claesson J, Andersson P, Johansson C, Lee R G, Prakash T P, Seth P P, Monia B P, Andersson S: Targeted delivery of antisense oligonucleotides to pancreatic beta-cells. Sci Adv 2018; 4:eaat3386
37. Templeman N M, Flibotte S, Chik J H L, Sinha S, Lim G E, Foster L J, Nislow C, Johnson J D: Reduced Circulating Insulin Enhances Insulin Sensitivity in Old Mice and Extends Lifespan. Cell Reports 2017; 20:451-463
38. Page M M, Skovsø S, Cen H, Chiu A P, Dionne D A, Hutchinson D F, Lim G E, Szabat M, Flibotte S, Sinha S, Nislow C, Rodrigues B, Johnson J D: Reducing insulin via conditional partial gene ablation in adults reverses diet-induced weight gain. The FASEB Journal 2018; 32:1196-1206

1. GC-globulin, or a variant thereof, for use in treating diabetes.

2. GC-globulin for the use of claim 1, wherein the diabetes is type 1 diabetes and/or type 2 diabetes.

3. A pharmaceutical composition for use in treating diabetes, comprising GC-globulin, or a variant thereof, and a pharmaceutically active carrier.

4. The pharmaceutical composition of claim 3, wherein the diabetes is type 1 diabetes and/or type 2 diabetes.

5. A method of treating diabetes in a subject in need thereof, comprising administering GC-globulin or a variant thereof to the subject.

6. The method of claim 4, wherein administering GC-globulin restores glucagon secretion in the subject

7. The method of claim 4, wherein administering GC-globulin increases insulin secretion in the subject.

8. The method of any one of claims 5 to 7, wherein the diabetes is type 1 diabetes and/or type 2 diabetes.

9. Use of GC-globulin or a variant thereof for the manufacture of a medicament for the treatment of diabetes.

10. The use of claim 9, wherein the diabetes is type 1 diabetes and/or type 2 diabetes.
